(19) United States
(12) Patent Application Publication
(10) Pub. No.: US 2026/0315257 A1
(43) Pub. Date: Oct. 15, 2026

(54) SUPPORT ARTICLE

(71) Applicant: 3M INNOVATIVE PROPERTIES COMPANY, St. Paul, MN (US)

(72) Inventors: Lawrence D. Fosler, River Falls, WI (US); Diane L. Emslander, Grant, MN (US)

(21) Appl. No.: 19/481,059

(22) PCT Filed: May 2, 2024
(86) PCT No.: PCT/IB2024/054278

§ 371 Date: Nov. 3, 2025

Related U.S. Application Data
(60) Provisional application No. 63/500,058, filed on May 4, 2023.

(51) Int. Cl. A61F 5/01

(52) U.S. Cl. CPC A61F 5/0109; A61F 5/0111; A61F 5/0118

(57) ABSTRACT

Provided is a joint support article. The joint support article includes a tubular body having two open ends and comprised of a knitted elastic material and having a hemmed edge along at least one of its open ends. A peripheral member coupled to an inner surface of the tubular body, and fully overlaps the hemmed edge. The peripheral member includes an elastic band having a tubular configuration complemental to that of the tubular body, and a frictional layer disposed on an inner surface of the elastic band to reduce a tendency of the peripheral member to slide against a wearer when the joint support article is worn.

### FIELD OF THE INVENTION

Provided are elastic support articles for muscle and joint wellness, which can be useful for example in supporting knees, elbows, and/or ankles.

### BACKGROUND

Elastic support articles provide support and/or mild compression to a stiff, weak or injured joint, such as the wrist, knee, elbow, or ankle. Support articles can have a contoured design and include precision straps to help keep the support in place during everyday use or other vigorous activities, while being easy to put on and take off. Support articles can be crafted from materials that can help keep the joint warm while allowing sweat to escape for breathable comfort.

### SUMMARY

One of the recurring problems with elastic knit supports is that they have a tendency to slide down and become misaligned relative to the joint that needs support.

Additionally, for some, skin contact along the hemmed edges of the elastic knit material can be uncomfortable when conventional support articles are worn. Incorporating an elastic band along the top or bottom edges of the support article provided with a frictional layer along its inner surface can help to provide comfort from the seam while helping it stay in place. These elastic bands can be added to the support along with other features.

In a first aspect, a joint support article is provided. The joint support article comprises: a tubular body having two open ends and comprised of a knitted elastic material, the tubular body having a hemmed edge along at least one of its open ends; a peripheral member coupled to an inner surface of the tubular body, wherein the peripheral member fully overlaps the hemmed edge and itself comprises: an elastic band having a tubular configuration complemental to that of the tubular body; and a frictional layer disposed on an inner surface of the elastic band to reduce a tendency of the peripheral member to slide against a wearer when the joint support article is worn.

In a second aspect, a method of making a joint support article is provided, comprising: providing a tubular body made from a knitted elastic material, the tubular body having a hemmed edge along at least one of its open ends; disposing a frictional layer onto an inner surface of an elastic band comprising a woven material to obtain a peripheral member; and coupling the peripheral member to an inner surface of the tubular body, wherein the peripheral member fully overlaps the hemmed edge and the peripheral member has a tubular configuration complemental to that of the tubular body.

### BRIEF DESCRIPTION OF THE DRAWINGS

FIG. 1 is an isometric view of a joint support article in its as-worn configuration;

FIG. 2 is a front elevational view of the joint support article of FIG. 1;

FIG. 3 is a side elevational view of the joint support article of FIGS. 1-2;

FIG. 4 is a side elevational view of the joint support article of FIGS. 1-3 with an internal component partially removed;

FIG. 5 is a side elevational view of the joint support article of FIGS. 1-4 turned inside out.

FIG. 6 is a top elevational view of the joint support article of FIGS. 1-5.

Repeated use of reference characters in the specification and drawings is intended to represent the same or analogous features or elements of the disclosure. It should be understood that numerous other modifications and embodiments can be devised by those skilled in the art, which fall within the scope and spirit of the principles of the disclosure. The figures may not be drawn to scale.

### DETAILED DESCRIPTION

As used herein, the terms “preferred” and “preferably” refer to embodiments described herein that can afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

As used herein and in the appended claims, the singular forms “a,” “an,” and “the” include plural referents unless the context clearly dictates otherwise. Thus, for example, reference to “a” or “the” component may include one or more of the components and equivalents thereof known to those skilled in the art. Further, the term “and/or” means one or all of the listed elements or a combination of any two or more of the listed elements. It is noted that the term “comprises”, and variations thereof do not have a limiting meaning where these terms appear in the accompanying description. Moreover, “a,” “an,” “the,” “at least one,” and “one or more” are used interchangeably herein. Relative terms such as left, right, forward, rearward, top, bottom, side, upper, lower, horizontal, vertical, and the like may be used herein and, if so, are from the perspective observed in the particular drawing. These terms are used only to simplify the description, however, and not to limit the scope of the invention in any way.

Reference throughout this specification to “one embodiment,” “certain embodiments,” “one or more embodiments” or “an embodiment” means that a particular feature, structure, material, or characteristic described relating to the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention.

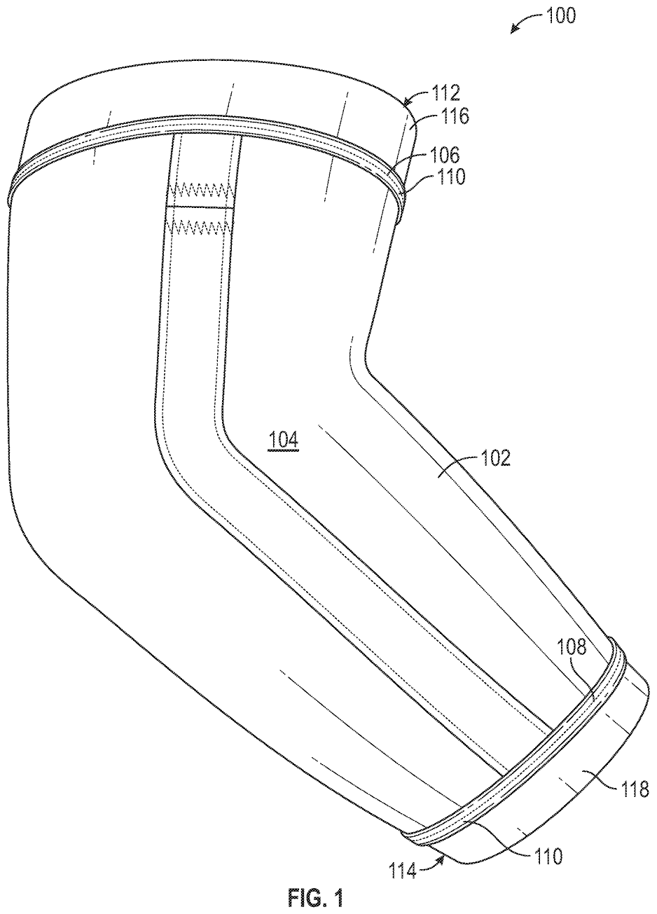

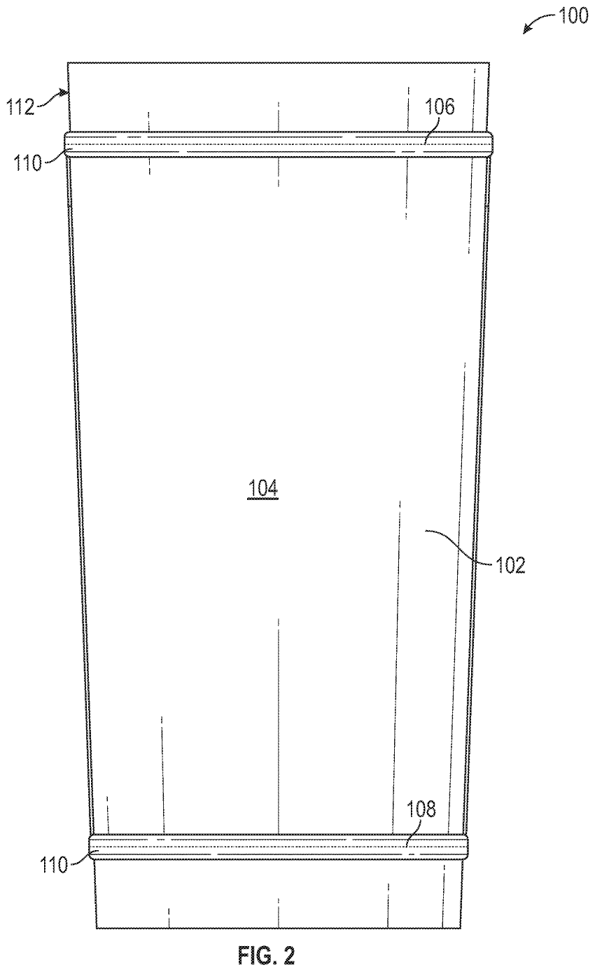

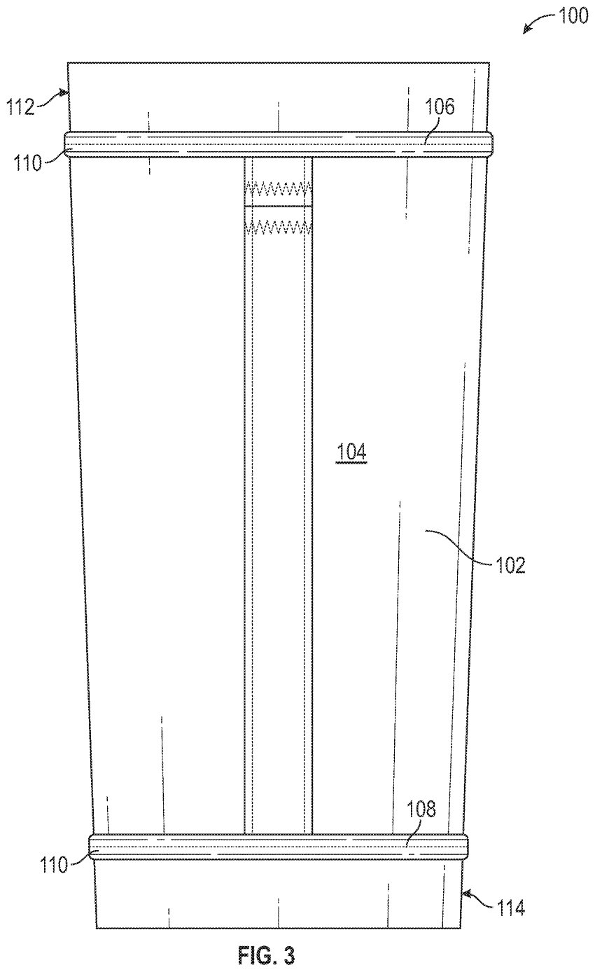

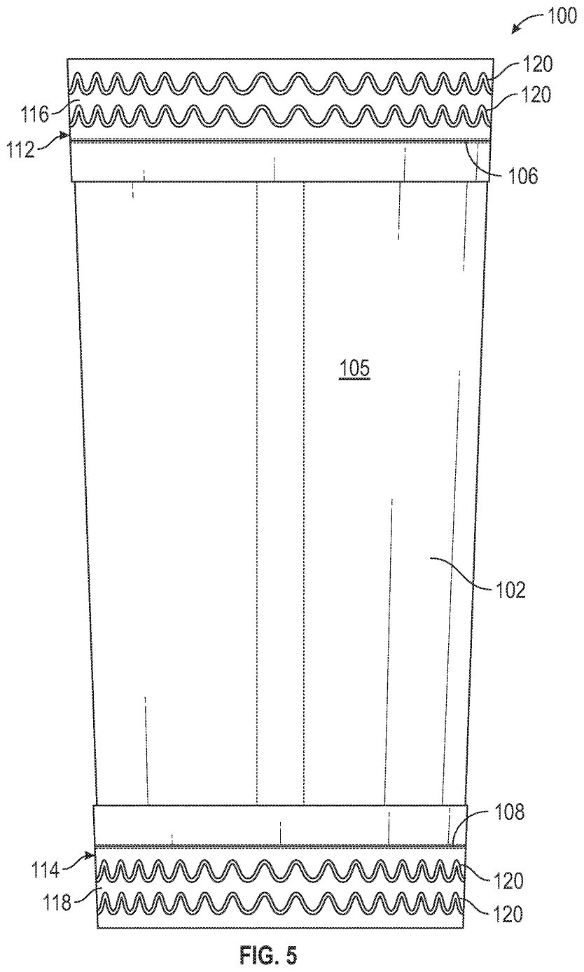

A joint support article according to one exemplary embodiment is shown in FIG. 1-6 and designated by the numeral 100. The article 100 includes a body 102 having a generally tubular shape. The body 102 has an outer surface 104 and an opposing inner surface 105 (visible in FIG. 5). Extending along the circuitous top and bottom of the body 102 are an upper peripheral edge 106 and lower peripheral edge 108. The body 102 is preferably comprised of an elastic material, such as a knitted elastic material.

The body 102 can have any suitable basis weight to provide a light, comfortable and low-profile fit against the joint of the wearer. The basis weight can be from 400 gsm to 850 gsm, from 440 gsm to 810 gsm, from 465 gsm to 775 gsm, or in some embodiments, less than, equal to, or greater than 400 gsm, 410, 420, 430, 440, 450, 460, 470, 480, 490, 500, 510, 520, 530, 540, 550, 600, 650, 700, 725, 750, 775, 800, 825, or 850 gsm.

Optionally and as shown, each of the upper and lower peripheral edges 106, 108 are hemmed edges. In the illustrated example, the hemmed upper and lower peripheral edges 106, 108 are provided by a binding 110. The binding 110 is a narrow strip of fabric that folds over a terminal edge of the knitted elastic material to provide the hems on the peripheral edges 106, 108, as shown in the figures. Alternatively, but not shown, the knitted elastic material can be folded upon itself along an open end of the tubular body 102 to provide the hems on peripheral edges 106, 108.

An upper peripheral member 112 is coupled to an inner surface 105 of the body 102 (visible in FIG. 5) and fully overlaps the upper peripheral edge 106. On the opposing end of the body article 100, a lower peripheral member 114 is also coupled to the inner surface 105 and fully overlaps the lower peripheral edge 108. In alternative embodiments, either the upper peripheral member 112 or the lower peripheral member 114 may be omitted without significantly comprising the functionality of the article 100. Such omission can provide a simpler and more cost-effective solution. Between the two, it is generally preferable for the upper peripheral member 112 (located above the lower peripheral member 114 when the article 100 is worn) to be retained to avoid slippage.

The peripheral members 112, 114 include respective elastic bands 116, 118 that are stitched to the body 102 and contact the wearer in encircling relation when the article 100 is worn. The elastic bands 116, 118 generally have tubular configurations complemental to that of the tubular body 102. While stitching is common, a weld or adhesive can also be useful in coupling the elastic bands 116, 118 and body 102 to each other. In some embodiments, one or both of the elastic bands 116, 118 are made from a woven material. Suitable woven materials can include polyester, nylon, elastane, cotton, rayon, hemp, and combinations thereof. FIG. 5 shows frictional layers 120 disposed on the inner surface of the elastic bands 116, 118. The frictional layers 120 reduces a tendency of the peripheral members 112, 114 to slide against the skin of a wearer, and assume any number of forms.

The stock material for the elastic bands 116, 118, which overlaps the body 102 along the inner surface, is generally thinner and more flexible than the material that used for the body 102. To provide a smoothly contoured contact surface against the skin along the inner surface 105, the basis weight of one or both elastic bands 116, 118 is preferably from 200 gsm to 850 gsm, from 255 gsm to 810 gsm, from 310 gsm to 775 gsm, or in some embodiments, less than, equal to, or greater than 200 gsm, 210, 220, 230, 240, 250, 255, 260, 270, 280, 290, 300, 310, 350, 400, 450, 500, 550, 600, 650, 700, 750, 775, 800, 810, or 850 gsm.

In the depicted embodiment, each frictional layer 120 takes the form of an extruded bead layer, which can be made from a silicone elastomer, urethane elastomer, combination thereof, or any other rubbery material that resists sliding against skin. The frictional layer 120 can include two or more non-intersecting extruded beads to provide a discontinuous layer as shown. Other possibilities can include a patterned layer consisting of a series of either continuous or discontinuous frictional features-e.g., replicated islands of a rubbery material disposed on the inner surface of the elastic bands.

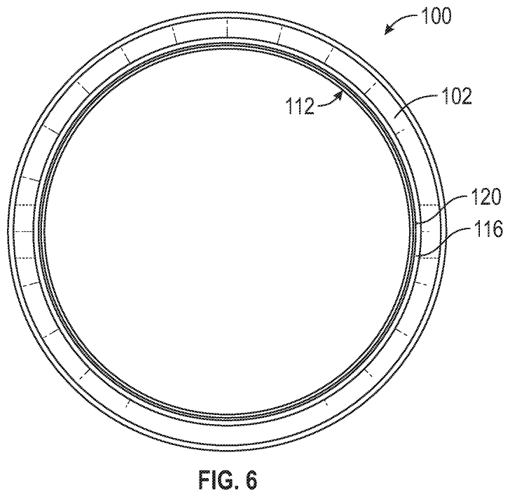

FIG. 6 shows the support article 100 as viewed along an axial direction, revealing the layered structure of the article 100. As shown, the peripheral member 112, including the elastic band 116 and frictional layer 120, is disposed along the inward-facing surfaces of the body 102. These components collectively stretch as a tight-fitting garment over a joint such as an ankle, elbow, or knee, with the joint generally having a cross-section greater than that of the unstretched support article 100. The elastic band 116 can have a configuration that enables compressive forces that are greater along the peripheral member 112 than along the body 102 alone, providing firm contact between the frictional layer 120 and the skin and reducing likelihood of slippage.

Optionally, and as shown in FIG. 5, the overlapping region between the peripheral member 112 and body 102 remains uncoated by the frictional layer 120. Since this overlapping region exerts even greater compressive force than either the body 102 or peripheral member 112 alone, locating the frictional layer 120 along portions of the elastic band 116 beyond the peripheral edge 106 can help moderate the level of compression and provide greater comfort to the wearer.

For improved comfort and functionality, it can be desirable for the body 102 to have a configuration that facilitates a four-way stretch, meaning the body 102 easily stretches along both the longitudinal direction of the article 100 as well as its circumferential direction (i.e., perpendicular to the longitudinal direction). This property is beneficial in allowing the body 102 to easily conform to the joint of the wearer when that joint is flexed. In contrast, it can be preferred for the elastic band 116, 118 of the peripheral member 112, 114 be made of a material that displays a two-way stretch and not a four-way stretch. In preferred embodiments, the two-way stretch is aligned along the circumferential direction with respect to the elastic bands 116, 118.

As used herein, “two-way stretch” can refer to a material, such as a fabric material, that elongates along a first direction to a much greater extent when stretched to its peak elongation than that along a second direction perpendicular to the first direction. As used herein, “peak elongation” represents the maximum length that a material can elastically stretch, expressed as a percentage of its original length when relaxed. For example, the peak elongation along its circumferential direction can be at least 50 percent, 60, 70, 80,90, 100, 110, 120, 150, 170, 200, 300, 400, or even 500 percent greater than the peak elongation along its longitudinal direction.

By contrast, “four-way” stretch describes a material, such as a fabric material, that elongates along its longitudinal direction when stretched to its peak uniaxial stress to a similar extent as that along its circumferential direction. For example, the peak elongation along the longitudinal and circumferential directions can differ from each other by a factor of at most 40 percent, 35, 30, 25, 20, 15, 10, or 5 percent, as defined relative to the smaller peak elongation value.

The body 102 can display a peak elongation δ along a given direction that is significantly greater than that of the elastic bands 116, 118 along the same direction. In some embodiments, the body 102 displays a peak elongation δ₁ along its circumferential direction and one or both elastic bands 116, 118 has a peak elongation δ₂ along its circumferential direction, where the ratio δ₁/δ₂ is in the range of from 1.3 to 1.5, or in some embodiments, less than, equal to, or greater than 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, or 1.5.

The body 102 preferably displays a tensile force at 50% of its peak elongation along its circumferential direction that provides a comfortable level of compression when stretched over a joint such as a knee, elbow, or ankle. The tensile force at 50% of peak elongation displayed by the body 102 can be from 4 N to 6 N, or in some embodiments, less than, equal to, or greater than 4 N, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, or 6 N. In various embodiments, one or both of the elastic bands 116, 118 can display a peak force along its circumferential direction from 900 N to 1100 N, or in some embodiments, less than, equal to, or greater than 900 N, 910, 920, 930, 940, 950, 960, 970, 980, 990, 1000, 1010, 1020, 1030, 1040, 1050, 1060, 1070, 1080, 1090, or 1100 N.

One or both peripheral members 112, 114 overlaps the inner surface 105 of the body 102 to an extent appropriate to reinforce the terminal portions of the body 102, to reduce sliding, and provide comfort to the wearer of the support article 100. The peripheral member can for example overlap from 2 percent to 20 percent, 3 percent to 17 percent, 5 percent to 15 percent, or in some embodiments, less than, equal to, or greater than 2 percent, 3, 4, 5, 6, 7, 8, 9, 10, 11, 12, 13, 14, 15, 16, 17, 18, 19, or 20 percent of the inner surface of the tubular body.

It can be advantageous for one or both peripheral members 112, 114 to extend significantly beyond the peripheral edges 106, 108 of the body 102 to taper the degree of compressive force applied along the areas of contact between the peripheral members 112, 114 and the wearer and provide greater comfort. It can be beneficial for 10 percent to 90 percent, from 25 percent to 75 percent, from 40 percent to 60 percent, or in some embodiments, less than, equal to, or greater than 10 percent, 15, 20, 25, 30, 35, 40, 45, 50, 55, 60, 65, 70, 75, 80, 85 or 90 percent of the peripheral members 112, 114 to extend beyond the respective peripheral edges 106, 108 of the body 102.

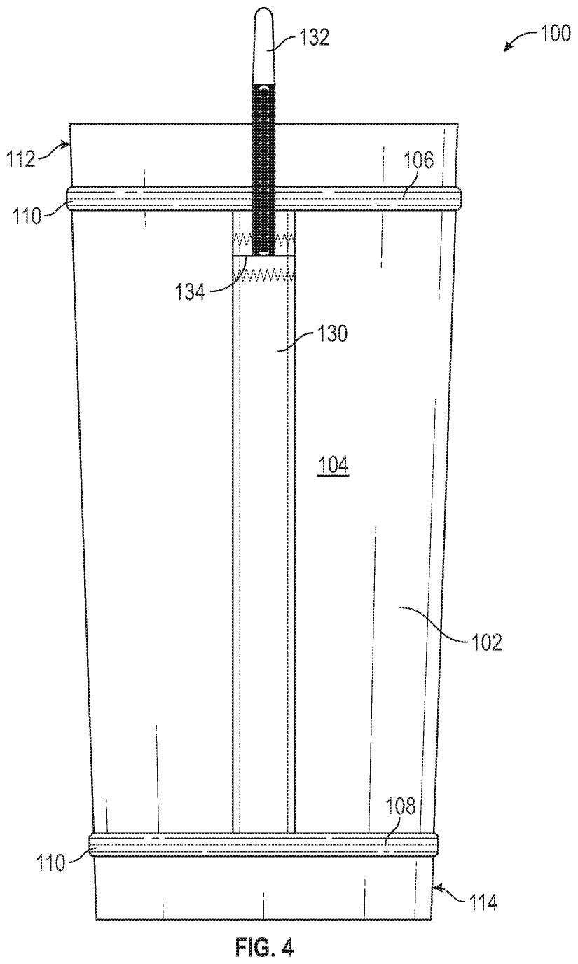

FIG. 4 shows the article 100 in a side view revealing an elongated pocket 130 stitched to its outer surface 104 as an optional feature. While only one elongated pocket 130 is shown in this figure, a second elongated pocket can be disposed on the opposing side of the article 100 such that the elongated pockets are symmetrically disposed on opposite sides of the joint when the article 100 is worn. Located partially within the elongated pocket 130 is a stiffening member 132, which is shown protruding through the offset slit 134.

The stiffening member 132 is generally flat and has a matching size and shape to fit snugly within the elongated pocket 130. The stiffening member 132 increases the stiffness to the body 102 and can be made from any combination of materials, commonly plastic, metal, or a combination thereof. If desired, the stiffening member 132 can have a flexural modulus that is directional, whereby the stiffening member 132 resists bending more along certain directions than along others. The slit 134 allows the stiffening member 132 to be easily removed or replaced by a user if desired. It is contemplated that use of provided support articles can include replacement of a first stiffening member with a second stiffening member with a different stiffness for therapeutic adjustments, as might be prescribed.

The aforementioned support articles can be made using any known method. An exemplary method entails providing a tubular body made from a knitted elastic material, the tubular body having a hemmed edge along at least one of its open ends, disposing a frictional layer onto an inner surface of an elastic band comprising a woven material to obtain a peripheral member, and coupling the peripheral member to an inner surface of the tubular body such that the peripheral member fully overlaps the hemmed edge and the peripheral member has a tubular configuration complemental to that of the tubular body. The order of steps need not be particularly restricted; for example, the step of coupling the peripheral member to an inner surface of the tubular body can occur before or after disposing the frictional layer onto the elastic band.

All cited references, patents, and patent applications in the above application for letters patent are herein incorporated by reference in their entirety in a consistent manner. In the event of inconsistencies or contradictions between portions of the incorporated references and this application, the information in the preceding description shall control. The preceding description, given in order to enable one of ordinary skill in the art to practice the claimed disclosure, is not to be construed as limiting the scope of the disclosure, which is defined by the claims and all equivalents thereto.

What is claimed is:

1. A joint support article comprising:
a tubular body having two open ends and comprised of a knitted elastic material, the tubular body having a hemmed edge along at least one of its open ends;
a peripheral member coupled to an inner surface of the tubular body, wherein the peripheral member fully overlaps the hemmed edge and itself comprises:
an elastic band having a tubular configuration complemental to that of the tubular body; and
a frictional layer disposed on an inner surface of the elastic band to reduce a tendency of the peripheral member to slide against a wearer when the joint support article is worn.

2. The joint support article of claim 1, wherein the tubular body comprises a binding that folds over a terminal edge of the knitted elastic material to provide the hemmed edge.

3. The joint support article of claim 1, wherein the knitted elastic material is folded upon itself along an open end of the tubular body to provide the hemmed edge.

4. The joint support article of claim 1, wherein the knitted elastic material has a configuration that facilitates a four-way stretch.

5. The joint support article of claim 1, wherein the elastic band has a configuration that facilitates a two-way stretch but not a four-way stretch.

6. The joint support article of claim 1, wherein the frictional layer is a patterned layer.

7. The joint support article of claim 6, wherein the patterned layer is a discontinuous layer.

8. The joint support article of claim 1, wherein the frictional layer comprises an extruded bead layer.

9. The joint support article of claim 8, wherein the extruded bead layer comprises a cured silicone elastomer, urethane elastomer, or combination thereof.

10. The joint support article of claim 1, wherein the knitted elastic material has an extent of elongation δ₁ when stretched to its peak uniaxial stress and the elastic band has a peak elongation δ₂ when stretched to its peak uniaxial stress, the ratio δ₁/δ₂ being in the range of from 1.3 to 1.5.

11. The joint support article of claim 1, wherein the peripheral member overlaps from 2 percent to 20 percent of the inner surface of the tubular body.

12. The joint support article of claim 11, wherein the peripheral member overlaps from 3 percent to 17 percent of the inner surface of the tubular body.

13. The joint support article of claim 12, wherein the peripheral member overlaps from 5 percent to 15 percent of the inner surface of the tubular body.

14. The joint support article of claim 1, wherein from 10 percent to 90 percent of the peripheral member extends beyond the hemmed edge of the tubular body.

15. The joint support article of claim 14, wherein from 25 percent to 75 percent of the peripheral member extends beyond the hemmed edge of the tubular body.

16. The joint support article of claim 15, wherein from 40 percent to 60 percent of the peripheral member extends beyond the hemmed edge of the tubular body.

17. The joint support article of claim 1, wherein the peripheral member is a first peripheral member and further comprising a second peripheral member coupled to a hemmed edge at an open end of the tubular body opposite that of the first peripheral member.

18. The joint support article of claim 1, wherein the elastic band comprises a woven material.

19. A method of making a joint support article comprising:
providing a tubular body made from a knitted elastic material, the tubular body having a hemmed edge along at least one of its open ends;
disposing a frictional layer onto an inner surface of an elastic band comprising a woven material to obtain a peripheral member; and
coupling the peripheral member to an inner surface of the tubular body, wherein the peripheral member fully overlaps the hemmed edge and the peripheral member has a tubular configuration complemental to that of the tubular body.
